(19) United States
(12) Plant Patent Application Publication
(10) Pub. No.: US 2017/0006756 P1
(43) Pub. Date: Jan. 5, 2017

(54) Pelargonium plant named 'KLEIZ14364'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/545,877

(22) Filed: Jul. 1, 2015

(51) Int. Cl. A01H 5/00

(52) U.S. Cl. PLT/328

(57) ABSTRACT

A new pelargonium plant particularly distinguished by having semi-double pink flowers with purple eyes, strong branching, and a compact habit, is disclosed.

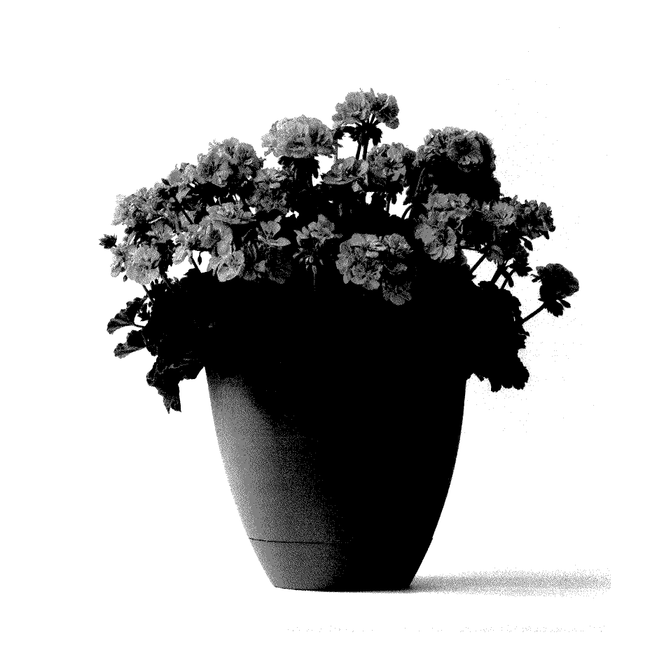

### GENUS AND SPECIES

Pelargonium sp.

### VARIETY DENOMINATION

‘KLEIZ14364’

### BACKGROUND OF THE NEW PLANT

The present invention comprises a new and distinct variety of pelargonium, botanically known as Pelargonium sp. and hereinafter referred to by the variety name ‘KLEIZ14364’. ‘KLEIZ 14364’ originated from a controlled-cross between the female pelargonium variety ‘Klegom’ (U.S. Plant Pat. No. 8,962) and the proprietary male pelargonium variety ‘PP 06 0086’ (unpatented).

In August 2008, the two pelargonium parental lines ‘Klegom’ and ‘PP 06 0086’ were crossed in Stuttgart, Germany and seeds were obtained. The seeds were sown and plants were grown for evaluation. A plant line was selected in August 2009 and subsequently named ‘KLEIZ14364’. In August 2009 ‘KLEIZ14364’ was first vegetatively propagated by vegetative cuttings. ‘KLEIZ14364’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. Semi-double pink flowers with purple eyes;

2. Strong branching; and

3. A compact habit.

### DESCRIPTION OF THE PHOTOGRAPH

This new pelargonium plant is illustrated by the accompanying photograph which shows an overall view of the plant. The photograph is of an approximately 4-month-old plant grown in Stuttgart, Germany under greenhouse conditions in June 2012. The colors shown are as true as can be reasonably obtained by conventional photographic procedures.

### DESCRIPTION OF THE NEW VARIETY

The following detailed descriptions set forth the distinctive characteristics of ‘KLEIZ14364’. The data which define these characteristics were collected from asexual reproductions carried out in Stuttgart, Germany. Data was collected on plants grown 3-months after planting in Stuttgart, Germany under greenhouse conditions in May 2015. Color references are to The R.H.S. Colour Chart of The Royal Horticultural Society of London (R.H.S.), 5^{th} edition (2007).
Classification:
   Family.—Geraniaceae.
   Botanical.—Pelargonium sp.
   Common.—pelargonium.
   Designation.—‘KLEIZ14364’.
Parentage:
   Female parent.—Pelargonium ‘Klegom’ (U.S. Plant Pat. No. 8,962).
   Male parent.—Proprietary pelargonium ‘PP 06 0086’ (unpatented).
Plant:
   Form.—Compact, bushy and round.
   Habit.—Upright.
   Height.—25.0 cm.
   Spread.—27.0 cm.
   Natural flowering season.—Spring to Autumn.
   Propagation type.—Vegetative cuttings.
   Time to produce a rooted cutting.—20 days.
   Time to bloom from propagation.—8 to 9 weeks.
   Lastingness of inflorescence on the plant.—6 days.
   Root description.—White and fine.
Stems:
   Quantity of stems per plant.—.
   Color.—RHS 137A.
   Surface texture.—Pubescent, short hairs.
   Stem form.—Round.
   Diameter.—0.8 cm.
   Length.—4.0 cm to 7.0 cm.
   Internode length.—1.0 cm to 2.0 cm.
   Anthocyanin.—Absent.
Leaves:
   Arrangement.—Alternate.
   Shape.—Transverse.
   Apex.—Round.
   Base.—Open.
   Margin.—Crenate.
   Surface texture.—Upper surface: Rough, pubescent, short hairs Lower surface: A little rough to smooth; in some areas pubescent with very short hairs.
   Length.—5.0 cm.
   Width.—7.0 cm.
   Color.—Upper surface: RHS 137A Lower surface: RHS 137C.
   Petiole.—Length: 4.0 cm to 6.0 cm Color: RHS 137A.
   Venation pattern.—Palmate.
   Venation color.—Upper surface: RHS 137 B Lower surface: RHS 137D.
Inflorescence:
   Lastingness of flowers on the plant.—5 to 6 days.
   Umbels.—Quantity of umbels per plant: 5 Length: 5.0 cm to 6.0 cm Diameter: 5.0 cm to 6.0 cm.
   Individual florets of the umbel.—Quantity of florets per umbel: 7 to 9 Depth: 1.5 cm Diameter: 3.0 cm.
   Fragrance.—Absent.
Flower buds:
   Cluster shape.—Umbelliferous.
   Cluster size.—Length: 3.0 cm Width: 3.0 cm.
   Surface texture.—Rough.
   Bud.—Height (cm): 1.0 cm to 1.5 cm Width (cm): 0.6 cm to 0.7 cm Shape: Elliptic Apex: Peaked Base: Blunt Margin: Entire Color: RHS 137B.
Petals:
   Quantity per flower.—5 to 6.
   Shape.—Drop-shaped.
   Apex.—Peaked.
   Base.—Attenuate.
   Margin.—Entire.
   Length.—2.0 cm.
   Width.—1.4 cm.
   Surface texture.—Upper surface: Smooth Lower surface: Smooth
   Color of lobes.—Upper lobes: Upper surface: RHS N66A, RHS67C, and RHS 69C Lower surface: RHS N67D Lower lobes: Upper surface: RHS N66A, RHS67C, and RHS 69C Lower surface: RHS 67D.
Petaloids:
   Quantity per flower.—2 to 3.
   Shape.—Moderate.
   Apex.—Round.
   Base.—Attenuate.
   Margin.—Smooth.
Sepals:
   Number per flower.—5.
   Shape.—Elliptic.
   Apex.—Peaked.
   Base.—Obtuse.
   Margin.—Entire.
   Surface texture.—Upper surface: Smooth Lower surface: Pubescent (moderate).
   Color.—Upper surface: RHS 137B Lower surface: RHS 137D.
   Length.—0.6 cm to 0.8 cm.
   Width.—0.3 cm to 0.5 cm.
Pedicel:
   Length.—1.5 cm to 2.0 cm.
   Diameter.—0.1 cm to 0.2 cm.
   Color.—RHS 137B.
   Surface texture.—Pubescent (moderate).
   Strength.—Medium.
Peduncle:
   Length.—15.0 cm to 18.0 cm.
   Diameter.—0.3 cm to 0.4 cm.
   Color.—RHS 137A.
   Surface texture.—Pubescent (moderate).
   Strength.—Medium-strong.
Reproductive organs:
   Stamens.—Quantity of stamens per flower: Moderate Anther length: 0.15 cm Filament length: 0.8 cm to 1.0 cm Filament color: RHS 49D Pollen amount: Moderate Pollen color: RHS 39D.
   Pistils.—Number: 1 Stigma color: RHS 66C Style length: 0.6 cm Style color: RHS 69D Ovary arrangement: Central Ovary color: RHS 145C Ovary diameter: 0.2 cm Ovary length: 0.3 cm.
   Fruit and seed set.—None observed.
   Disease and pest/insect resistance.—None observed.

### COMPARISON WITH PARENTAL LINES AND KNOWN VARIETY

‘KLEIZ14364’ is distinguished from its parents as described in Table 1:

TABLE 1
Comparison with Parental Lines

| | | Female parent | Male Parent |
|---|---|---|---|
| Trait | ‘KLEIZ14364’ | ‘Klegom’ | ‘PP 06 0086’ |
| Branching | Strong | Medium | Medium |
| Flower color | Pink with purple | Salmon pink with | Dark red |
| | eyes | purple eyes | |
| Earliness of | Very early | Early | Mid-early |
| flowering | | | |

‘KLEIZ14364’ can be compared to the commercial variety Sunrise Mona Lisa (unpatented). Differences between the two varieties are described in Table 2:

TABLE 2
Comparison with Similar Variety

| | | Commercial line |
|---|---|---|
| Trait | ‘KLEIZ14364’ | Sunrise Mona Lisa |
| Growth habit | Compact | Medium |
| Flower color | Pink with purple eyes | Blue pink with purple eyes |
| Flower type | Semi-double | Double |

We claim:

1. A new and distinct variety of pelargonium plant named ‘KLEIZ14364’ as illustrated and described herein.
